(19) United States
(12) Patent Application Publication
(10) Pub. No.: US 2003/0129160 A1
(43) Pub. Date: Jul. 10, 2003

(54) Use of Interleukin-6

(76) Inventors: John-Olov Jansson, Donso (SE); Kaj Stenlof, Torslanda (SE); Ville Wallenius, Goteborg (SE); Kristina Wallenius, Goteborg (SE)

Correspondence Address: BENTON S. DUFFETT, JR., BURNS, DOANE, SWECKER & MATHIS, L.L.P., P.O. Box 1404, Alexandria, VA, 22313-1404, US

(21) Appl. No.: 10/040,335

(22) Filed: Jan. 9, 2002

(51) Int. Cl. A61K 38/20

(52) U.S. Cl. 424/85.2

(57) ABSTRACT

This invention relates to a method for chronic treatment of obesity wherein a pharmaceutically effective amount of a substance that upon administration to a patient without complete IL-6 deficiency will lead to an increased level of an IL-6 receptor agonist is administered to said patient for reducing adipose tissue mass.

Furthermore, the invention relates to the use of a substance that upon administration to a patient without complete IL-6 deficiency will lead to an increased level of an IL-6 receptor agonist for the production of a medicinal product for reducing adipose tissue mass for chronic treatment of obesity.

### TECHNICAL FIELD OF THE INVENTION

The present invention relates to a new medicinal product and a new method for treatment of obesity.

### BACKGROUND ART

Obesity and obesity-related disorders are among the leading causes of illness and mortality in the developed world (Kopelman PG, 2000, “Obesity as a medical problem”, Nature 404: 635-43). Parts of the brain, including specific regions of the hypothalamus and the brain stem, are involved in the regulation of feeding and body fat mass (Friedman J M, Halaas J L, 1998, “Leptin and the regulation of body weight in mammals”, Nature 395: 763-70; Schwartz M W, Woods S C, Porte D Jr, Seeley R J, Baskin D G, 2000, “Central nervous system control of food intake”, Nature Apr 6; 404 (6778): 661-71). The regulation of feeding and body mass by the hypothalamus is influenced e.g. by the adipose tissue derived hormone leptin. It is well recognized that circulating leptin affects metabolic functions and body composition via the hypothalamus (Friedman J M, Halaas J L, 1998, “Leptin and the regulation of body weight in mammals”, Nature 395: 763-70).

Leptin levels in blood reflect adipose tissue mass and leptin treatment can reverse obesity in leptin-deficient mice, but not in non-leptin deficient individuals, that have high endogenous leptin levels (Friedman J M, Halaas J L, 1998, “Leptin and the regulation of body weight in mammals”, Nature 395: 763-70; Flier J S, Maratos-Flier E, 1998, “Obesity and the hypothalamus: novel peptides for new pathways”, Cell 92: 437-40), Therefore, a physiological function of a hormone is not necessarily accompanied by a clear-cut therapeutic potential.

Interleukin-6 (IL-6) is well known for its effects on immune functions and is released from Immune cells during inflammation (Van Snick J, 1990, “Interleukin-6: an overview”, Annu Rev Immunol 8; 253-78). In the absence of inflammation, circulating IL-6 is to a large part derived from adipose tissue (Mohamed-Ali V, Goodrick S, Rawesh A, Katz D R, Miles J M, Yudkin J S, Klein S, Coppack S W, 1997, “Subcutaneous adipose tissue releases interleukin-6, but not tumor necrosis factor-alpha, in vivo”, J Clin Endocrinol Metab 82: 4196-200) and IL-6 levels in blood correlate to adipose tissue mass (Mohamed-Ali V, Goodrick S, Rawesh A, Katz D R, Miles J M, Yudkin J S, Klein S, Coppack S W, 1997, “Subcutaneous adipose tissue releases interleukin-6, but not tumor necrosis factor-alpha, in vivo”, J Clin Endocrinol Metab 82: 4196-200; Vgontzas A N, Papanicolaou D A, Bixler E O, Kales A, Tyson K, Chrousos G P, 1997, “Elevation of plasma cytokines in disorders of excessive daytime sleepiness: role of sleep disturbance and obesity”, J Clin Endocrinol Metab 82: 1313-6), in a way similar to leptin. Both short term and long term changes in food intake increase serum levels of IL-6 (Orban Z, Remaley A T, Sampson M, Trajanoski Z, Chrousos G P, 1999, “The differential effect of food intake and beta-adrenergic stimulation on adipose-derived hormones and cytokines in man”, J Clin Endocrinol Metab 84: 2126-33; Bastard J P, Jardel C, Bruckert E, Blondy P, Capeau J, Laville M, Vidal H, Hainque B, 2000, “Elevated levels of interleukin 6 are reduced in serum and subcutaneous adipose tissue of obese women after weight loss”, J Clin Endocrinol Metab 85: 3338-42). Further, IL-6 and its receptor are expressed in discrete hypothalamic nuclei that have an established role in the regulation of metabolism and body composition (Schobitz B, de Kloet E R, Sutanto W, Holsboer F, 1993, “Cellular localization of interleukin 6 mRNA and interleukin 6 receptor mRNA in rat brain”, Eur J Neurosci 5: 1426-35; Shizuya K, Komori T, Fujiwara R, Miyahara S, Ohmori M, Nomura J, 1998, “The expressions of mRNAs for interleukin-6 (IL-6) and the IL-6 receptor (IL-6) in the rat hypothalamus and midbrain during restraint stress”, Life Sci 62: 2315-20). Therefore, several non-immune organs that have an established role in the regulation of metabolism and body composition also produce IL-6.

The present inventors recently demonstrated that interleukin-6 (IL-6) deficient mice developed obesity and obesity related metabolic disorders and that these effects could partly be reversed by IL-6 replacement (Wallenius v, Wallenius K, Ahrén B, Rudling M, Carlsten H, Dickson S L, Ohlsson C, Jansson J-O, 2002, “Interleukin-6 gene knockout causes mature-onset obesity in mice”, Nature Medicine 8: 75-79 and international patent application WO 01/03725). However, in this study no effect of IL-6-treatment in the non-IL-6-deficient mice was observed.

Recently Metzger et al reported reduced body fat in mice that carried an IL-6 secreting tumor for 18 days compared to pair-fed mice bearing a non-secreting tumor (Metzger S, Hassin T, Barash V, Pappo O, Chajek-Shaul T, 2001, “Reduced body fat and increased hepatic lipid synthesis in mice bearing interleukin-6-secreting tumor”, Am J Physiol Endocrinol Metab 281: E957-65). However, these results may be difficult to interpret. At the end of this study when body composition was analyzed, serum IL-6 levels were 40 ng/ml, which is very high and similar to or higher than the levels seen during bacterial infection and sepsis (Metzger S, Goldschmidt N, Barash V, Peretz T, Drize O, Shilyansky J, Shiloni E, Chajek-Shaul T, 1997, “Interleukin-6 secretion in mice is associated with reduced glucose-6-phosphatase and liver glycogen levels”, Am J Physiol 273: E262-7). Moreover, the effects of tumor burden and secretion of other factors from the tumor are likely to be permissive for the effect of these very high doses of IL-6 on adipose tissue mass.

Ciliary neurotrophic factor (CNTF) is a cytokine that acts via similar receptor mechanisms as IL-6. The ligand binding parts of the CNTF receptor and the IL-6 receptor both bind to the same signal transducing subunit (gp130) (Gadient R A, Patterson P H, 1999, “Leukemia inhibitory factor, Interleukin 6, and other cytokines using the GP130 transducing receptor: roles in inflammation and injury”, Stem Cells 17: 127-37; Hirano T, 1998, “Interleukin 6 and its receptor: ten years later”, Int Rev Immunol 16: 249-84). Unlike IL-6, CNTF does not act on neurons in the arcuate nucleus (Bjorbaek C, Elmquist J K, ElHachimi K, Kelly J, Ahima R S, Hileman S, Flier J F, 1999, “Activation of SOCS-3 messenger ribonucleic acid in the hypothalamus by ciliary neurotrophic factor” Endocrinology 140: 2035-43) Low doses of CNTF, which do not cause acute phase reaction or fever, have been shown to reduce body fat in mice with diet induced obesity (Lambert P D, Anderson K D, Sleeman M W, Wong V, Tan J, Hijarunguru A, Corcoran T L, Murray J D, Thabet K E, Yancopoulos G D, Wiegand S J, 2001, “Ciliary neurotrophic factor activates leptin-like pathways and reduces body fat, without cachexia or rebound weight gain, even in leptin resistant obesity”, Proc Natl Acad Sci USA 98: 4652-7, Gloaguen I, Costa P, Demartis A, Lazzaro D, Di Marco A, Graziani R, Paonessa G, Chen F, Rosenblum C I, Van der Ploeg L H, Cortese R, Ciliberto G, Laufer R, 1997, “Ciliary neurotrophic factor corrects obesity and diabetes associated with leptin deficiency and resistance”, Proc Natl Acad Sci USA 94: 6456-61) and clinical trials with a CNTF analogue have shown that CNTF can reduce body weight also in humans (Bray G A, Tartaglia L A, 2000, “Medicinal strategies in the treatment of obesity”, Nature 404: 672-7). However, unlike IL-6, CNTF is not released systemically during conditions associated with cachexia and loss of lean body mass. Therefore, it is surprising that CNTF can exert beneficial effects on body fat without causing cachexia. Chronic treatment with high doses of CNTF caused protein degradation and anorexia (Espat N J, Auffenberg T, Rosenberg J J, Rogy M, Martin D, Fang C H, Hasselgren P O, Copeland E M, Moldawer L L, 1996, “Ciliary neurotrophic rotrophic factor is catabolic and shares with IL-6 the capacity to induce an acute phase response”, Am J Physiol 271: R185-90).

It has been shown that single injections of high doses of IL-6, given peripherally, can acutely increase energy expenditure in humans (Tsigos C, Papanicolaou D A, Defensor R, Mitsiadis C S, Kyrou I, Chrousos G P, 1997 “Dose Effects of Recombinant Human Interleukin-6 on Pituitary Hormone Secretion and Energy Expenditure”, Neuroendocrinology 66: 54-62). In addition, the present inventors and others have reported that intracerebroventricular (ICV) injection of a low dose of IL-6, but not peripheral treatment with the same dose, acutely increases energy expenditure by single injections (Wallenius V, Wallenius K, Ahrén B, Rudling M, Carlsten H, Dickson S L, Ohlsson C, Jansson J-O, 2002, “Interleukin-6 gene knockout causes mature-onset obesity in mice”, Nature Medicine 8: 75-79; Rothwell N J, Busbridge N J, Lefeuvre R A, Hardwick A J, Gauldie J, Hopkins S J, 1991, “Interleukin-6 is a centrally acting endogenous pyrogen in the rat”, Can J Physiol Pharmacol 69: 1465-9). However, an acute increase in energy expenditure may not be of therapeutic value, since a stimulatory effect of a single injection of IL-6 on energy expenditure may be accounted for by enhanced body temperature (Rothwell N J, Busbridge N J, Lefeuvre R A, Hardwick A J, Gauldie J, Hopkins S J, 1991, “Interleukin-6 is a centrally acting endogenous pyrogen in the rat”, Can J Physiol Pharmacol 69: 1465-9). Moreover, an acute effect is often not accompanied by a clinically relevant chronic effect. It has been reported that ICV treatment with single injections of IL-6 can suppress 2-h food intake (Plata-Salaman C R, 1996, “Anorexia induced by activators of the signal transducer gp 130”, Neuroreport 7: 841-4), but not 24-h food intake (Plata-Salaman C R, Sonti G, Borkoski J P, Wilson C D, French-Mullen J M b, 1996, “Anorexia induced by chronic central administration of cytokines at estimated pathophysiological concentrations”, Physiol Behav 60: c867-75).

Chronic elevated levels of IL-6, as in transgenic mouse models, have been shown to cause muscle atrophy (Tsujinaka T, Ebisui C, Fujita J, Kishibuchi M, Morimoto T, Ogawa A, Katsume A, Ohsugi Y, Kominami E, Monden M, 1995, “Muscle undergoes atrophy in association with increase of lysosomal cathepsin activity in interleukin-6 transgenic mouse”, Biochem Biophys Res Commun 207: 168-74). The muscle atrophy seen in IL-6 transgenic mice has been assumed to mimic the muscle wasting during severe infections and cancer (Matthys P, Billiau A, 1997, “Cytokines and cachexia”, Nutrition 13: 763-70). Stunted growth is observed in some IL-6 transgenic mice and this effect is thought to be due to decreased serum IGF-I levels (De Benedetti F, Alonzi T, Moretta A, Lazzaro D, Costa P, Poli V, Martini A, Ciliberto G, Fattori E, 1997, “Interleukin 6 causes growth impairment in transgenic mice through a decrease in insulin-like growth factor-I. A model for stunted growth in children with chronic inflammation”, J Clin Invest 99: 643-50). Moreover, IL-6 given peripherally at high doses to normal non-IL-6-deficient individuals causes deleterious effects, e.g. on blood lipids (Greenberg, AS et al, 1992, “Interleukin 6 reduces lipoprotein lipase activity in adipose tissue of mice in vivo and in 3T3-L1 adipocytes: a possible role for interleukin 6 in cancer cachexia”, Cancer Res 52: 4113-6; Nonogaki, K et al, 1995, “Interleukin-6 stimulates hepatic triglyceride secretion in rats”, Endocrinology 136: 2143-9) and blood glucose (Tsigos, C et al, 1997, “Dose-dependent effects of recombinant human interleukin-6 on glucose regulation” [see comments], J Clin Endocrinol Metab 82, 4167-70). Elevated peripheral IL-6 levels, as seen in obesity (Mohamed-Ali V, Goodrick S, Rawesh A, Katz D R, Miles J M, Yudkin J S, Klein S, Coppack S W, 1997, “Subcutaneous adipose tissue releases interleukin-6, but not tumor necrosis factor-alpha, in vivo”, J Clin Endocrinol Metab 82: 4196-200; Vgontzas A N, Papanicolaou D A, Bixler E O, Kales A, Tyson K, Chrousos G P, 1997, “Elevation of plasma cytokines in disorders of excessive daytime sleepiness: role of sleep disturbance and obesity”, J Clin Endocrinol Metab 82: 1313-6) seem to be correlated with increased blood lipids, blood glucose and mortality. Therefore, it has been suggested that suppression of IL-6 could have beneficial effects in humans (Staels, B et al, 1998, “Activation of human aortic smooth-muscle cells is inhibited by PPAR-alpha but not by PPARgamma activators” Nature 393, 790-3; McCarty, M F, 1999, “Interleukin-6 as a central mediator of cardiovascular risk associated with chronic inflammation, smoking, diabetes, and visceral obesity: down-regulation with essential fatty acids, ethanol and pentoxifylline” Med Hypotheses 52, 465-77; Yudkin, J S, Kumari, M, Humphries, S E & Mohamed-Ali, V, 1999, “Inflammation, obesity, stress and coronary heart disease: is interleukin-6 the link?” Atherosclerosis 148: 209-14; Ershler, W B & Keller, E T, 2000, “Age-associated increased interleukin-6 gene expression, late-life diseases, and frailty” Annu Rev Med 51: 245-70).

### SUMMARY OF THE INVENTION

The aim of the present invention is to provide new medical products and methods for treatment of obesity.

More precisely, the invention relates to the use of a substance that upon administration to a patient without complete IL-6 deficiency will lead to an increased level of an IL-6 receptor agonist for the production of a medicinal product for reducing adipose tissue mass for chronic treatment of obesity.

Furthermore, the invention relates to a method for chronic treatment of obesity wherein a pharmaceutically effective amount of a substance that upon administration to a patient without complete IL-6 deficiency will lead to an increased level of an IL-6 receptor agonist is administered to said patient for reducing adipose tissue mass.

### DETAILED DESCRIPTION OF THE INVENTION

In the research work leading to the present invention it was found that chronic treatment with centrally administered IL-6 selectively can decrease body fat in non-IL-6-deficient rats fed a high-fat diet without causing signs of acute phase reaction or illness. The inventors have found that the central nervous system (CNS), e.g. the hypothalamus which contains IL-6 receptors, is a target for the adipostatic effects of IL-6.

The invention thus relates to medicinal products comprising a substance that upon administration to a patient without complete IL-6 deficiency will lead to an increased level of an IL-6 receptor agonist. Preferably, said administration leads to an increased level of IL-6 in the cerebrospinal fluid (CSF).

Said substance may be an IL-6 receptor agonist. A preferred example of such an agonist is IL-6. It is also possible to use functionally equivalent analogues of IL-6. Further, it is possible to use a naturally occurring agonist, such as IL-6, as well as a synthetically produced agonist, such as an IL-6 mimetic. Examples of synthetically produced IL-6 receptor agonists are given in U.S. Pat. No. 550,61,07 (Cunningham et al), U.S. Pat. No. 589,19,98 (Rocco et al), and U.S. Pat. No. 591,41,06 (Gennaro et al). Said substance may also be a substance that upon administration will lead to the release of an endogenous occurring IL-6 receptor agonist, preferably IL-6.

The expression “IL-6 receptor agonist” used herein relates to all substances that bind to and activate the same receptor proteins as IL-6.

The expression “functionally equivalent analogue” used herein relates to any substance that is structurally similar to IL-6 and has essentially the same pharmacological and/or therapeutical effects.

The term “patient” used herein relates to any human or non-human mammal in need of treatment with the medicinal product or method according to the invention.

Patients particularly suitable for treatment according to the invention are patients without complete IL-6 deficiency. By a patient without complete IL-6 deficiency is meant a patient who possesses a functional IL-6-gene and is capable of releasing endogenous IL-6. Preferably, the patient has normal levels of IL-6 in serum. By a patient having normal levels of IL-6 in serum is meant a patient having above the 5^{th} percentile level of IL-6 found in serum in healthy individuals. Patients particularly suitable for treatment according to the invention are patients having IL-6 levels in the CSF, which are lower than the average IL-6 levels found in the CSF in healthy individuals.

The term “treatment” used herein relates to both treatment in order to cure or alleviate a disease or a condition, and to treatment in order to prevent the development of a disease or a condition. By “chronic treatment” is meant treatment that continues for more than two weeks.

The medicinal product and the method according to the invention are suitable for treatment of different pathological disturbances of regulation of body adipose tissues. More precisely, the medicinal product and the method according to the invention are suitable for treatment of obesity and overweight by reducing adipose tissue mass.

Obesity includes visceral or general obesity that is due to genetic predisposition, a condition sometimes described as the thrifty genotype. Obesity caused by lifestyle and environment, such as lack of exercise, or diets with high caloric content or high fat content, can also be treated as described herein. The medicinal product and the method according to the invention could also be used to enhance the effects of exercise and/or diet. Obesity is often associated with resistance to leptin treatment.

The reduction in adipose tissue mass according to the invention preferably results in a weight reduction that is larger than 5% of body weight at the start of treatment.

The medicinal product or pharmaceutical composition or pharmaceutical preparation according to the invention may also comprise other substances, such as an inert vehicle, or pharmaceutical acceptable adjuvants, carriers, preservatives etc., which are well known to persons skilled in the art.

Said substance according to the invention is preferably formulated in a form enabling passage of said IL-6 receptor agonist through the blood-brain barrier, i.e. passage from the blood circulation to the CSF and the neurons in the CNS.

Said substance can be administered subcutaneously, intramuscularely, intravenously, intranasally or orally.

The substance according to the invention is preferably administered in a dose of 20 ng to 200 μg per kg body weight.

The invention also relates to use of a substance that upon administration to a patient without complete IL-6 deficiency will lead to an increased level of an IL-6 receptor agonist for the production of a medicinal product for reducing adipose tissue mass for chronic treatment of obesity.

Furthermore, the invention relates to a method for chronic treatment of obesity wherein a pharmaceutically effective amount of a substance that upon administration to a patient without complete IL-6 deficiency will lead to an increased level of an IL-6 receptor agonist is administered to said patient for reducing adipose tissue mass.

In the method according to the present invention, a “pharmaceutically active amount” of the substance is used. This expression relates to a dose of the substance that will lead to the desired pharmacological and/or therapeutic effect. The desired pharmacological and/or therapeutic effect is, as stated above, to cure or alleviate different pathological disturbances of regulation of body adipose tissues, leading to obesity, i.e. treatment of obesity and overweight by reducing adipose tissue mass.

Furthermore, it is possible to combine the treatment according to the invention with other conventional pharmacological treatments of obesity. The substance according to the invention may thus be administered in combination with other conventional pharmaceuticals used to treat obesity.

The invention will now be further explained in the following examples. These examples are only intended to illustrate the invention and should in no way be considered to limit the scope of the invention.

### BRIEF DESCRIPTION OF THE DRAWINGS

In the examples below reference is made to the accompanying drawings, where FIGS. 1-2 concern experiments performed on rats and FIG. 3 concerns experiments performed on humans, on which:

FIG. 1A shows the changes in body weight during two weeks of ICV treatment with IL-6 (0.4 μg/day) or saline to male rats on a high fat diet. FIG. 1B shows the body weights before (day 0) and after (day 14) two weeks of ICV treatment with saline or IL-6 (b). *P<0.05, **P<0.01 vs. corresponding control.

FIG. 2 shows dissected fat pads and serum leptin. Three intra-abdominal fat pads (gonadal (Gon), retroperitoneal (Ret) and mesenteric (Mes)) and the inguinal (Ing) fat pad (a subcutaneous fat pad in the groin) were dissected. FIG. 2A shows the total weight of the dissected fat pads after two weeks of ICV treatment with saline or IL-6 (0.4 μg/day). FIG. 2B shows a comparison between the relative weight of the different dissected fat pads (% of body weight) after saline and IL-6 treatment. FIG. 2C shows the leptin levels before and after two weeks of ICV treatment with saline or IL-6 treatment. A and B*P<0.05, vs. corresponding control, C**P<0.01 vs. before IL-6 treatment.

FIG. 3A shows IL-6 levels in CSF vs. total body fat in humans. Total body fat was measured by DXA. FIG. 3B shows the IL-6 levels in CSF vs. subcutaneous thigh adipose tissue measured with computed tomography (CT) at a level of half way between the hip and the knee. FIG. 3C shows IL-6 levels in CSF vs. IL-6 levels in serum from the same individual.

### EXAMPLES

Experiments Performed on Rats:

Animals

Male Wistar rats (Charles River, Margate, UK) were maintained under standardized environmental conditions, i.e. 24-26° C., 50-60% relative humidity, artificial lighting at 06.00-19.00 h, with water and pelleted food ad libitum. The rats were placed on high fat, Dairy Butter Diet (ICN Biomedicals, Costa Mesa, Calif., USA) one-week after arriving to the animal facility and were kept on the diet throughout the study.

Intracerebroventricular (ICV) Cannulation

The rats were anaesthetized by intraperitoneal injection of tribromoethanol/amylhydrate (avertin, 10 ml/kg) and placed in a stereotactic frame with the nose bar set at 3 mm below the interaural line. Permanent 28 gauge stainless steel guide cannulae (Plastics-One, Roanoke, Va., USA) were positioned in the lateral ventricle using stereotactic co-ordinates (0.6 mm posterior to the bregma, 1.6 mm lateral to midline and 4.0 mm below the outer surface of the skull). Guide cannulae were held in position by dental cement attached to three stainless steel screws driven into the skull. A stainless steel obdurator (Plastics One, Roanoke, Va., USA), which protruded 0.5 mm beyond the guide cannula, was inserted to maintain cannula patency. After one-week recovery from the surgery, the rats were handled on a daily basis and injected with saline. Prior to the study, human angiotensin II (Sigma, Poole, Dorset, UK) was injected ICV (150 ng per rat; volume=10 μl) to confirm the correct position of the cannula. Rats, which showed a sustained drinking response within two minutes following injection of angiotensin II, were included in the study.

Study

Two weeks after ICV cannulation, the rats were given a daily ICV injection of either rat recombinant IL-6 (0.4 μg/day, lot # 10786 J207, PeproTech EC LTD, London, UK) or an equal volume (10 μl) saline (Animal Care limited, York, UK) for two weeks. Measurement of specific activity of this lot of IL-6 by the in vitro bioassay (SBH Sciences, Inc, Natick, Mass., USA) showed an ED₅₀ ≦0.01 ng/ml. Food intake and body weight was monitored every day. At the end of the study the rats were terminally anaesthetised (barbiturate 70 mg/kg; Rhone Merieux, Inc., Harlow, Essex, UK) and blood was collected by cardiac puncture. Three intra-abdominal fat pads (gonadal, retroperitoneal and mesenteric) and one subcutaneous fat pad (inguinal) as well as several other organs were dissected and weighed. The rats were not treated with IL-6 or saline the day the study ended.

Serum Analysis

A blood sample was collected from the tail vein of conscious rats on the day before the study started. At the end of the study, blood was collected by cardiac puncture from anaesthetized rats. The blood samples were immediately placed on ice and later centrifuged to obtain serum. Serum samples were kept at −80° C. for future analysis. Serum leptin and insulin were assayed using enzyme linked immunosorbent assays (Crystal Chem Inc, Chicago, Ill., USA). Glucose was measured using reagents from Sigma Diagnostics (Infinity glucose reagent; Sigma diagnostics Inc, St Louis, Mo., USA). Insulin-like growth factor I (IGF-I) was analyzed by radioimmunoassay (Mediagnost Gmbh, Tubingen, Germany). Murine Serum Amyloid A (SAA) was analyzed by enzyme linked immunosorbent assay (Tridelta Development Ltd, Bray, Ireland). Corticosterone was analyzed by radioimmunoassay (ImmunoChem ICN Biomedicals Inc, Calif., USA).

Statistical Analysis

Values are given as mean ± standard error of the mean (SEM). Comparisons between two groups of rats were made by unpaired Students t test. Comparisons within each group before and after treatment were made by paired Students t test.

### Example 1

(Body Weight)

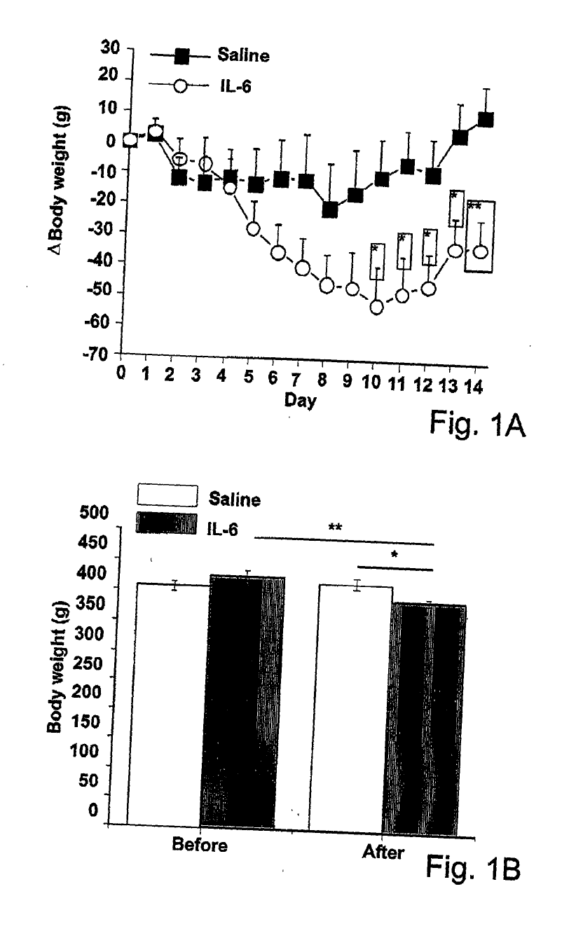

In this example, the effects of ICV injections of IL-6 on the body weight were studied. Rats were given daily ICV injections of IL-6 or saline from day 1 to day 14 and body weight was monitored every day. At day 5 of treatment the body weights of the IL-6 treated group started to decrease and deviate from the saline treated group I, as evident from FIG. 1A. On day 10, the body weights were significantly decreased in the IL-6 treated group compared to the saline treated group and this difference remained throughout the study. At the end of the study the body weights of the saline treated rats had increased by 2.2% whereas body weights of the IL-6 treated rats had decreased by 8.4%, as seen in FIG. 1B.

### Example 2

(Dissected Fat Pads and Serum Leptin)

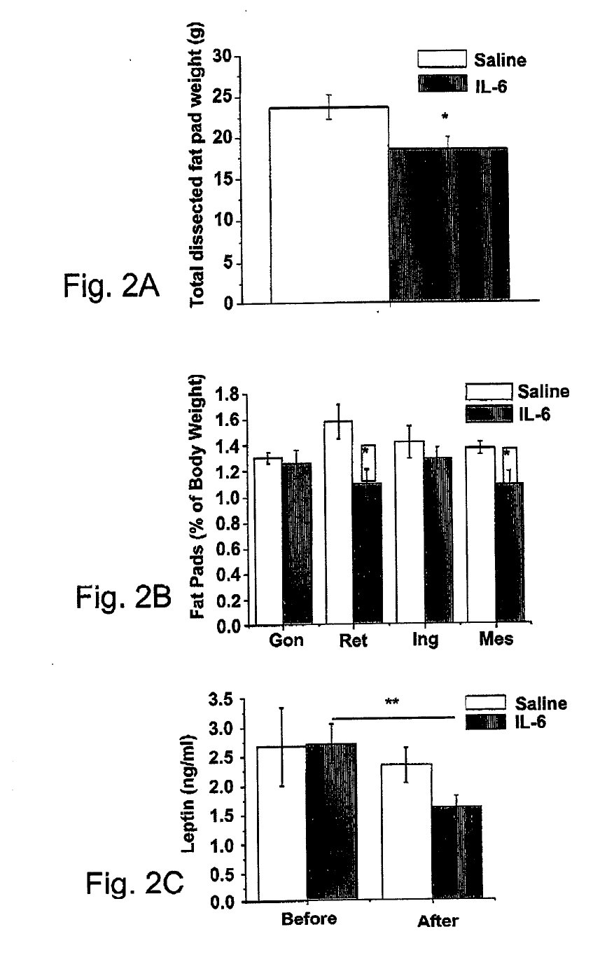

Several different fat pads were dissected and weighed at the end of the study. As can be seen in FIG. 2A, the total weight of all dissected fat pads was significantly lower in the IL-6 treated compared to the saline treated group. The relative weights of the mesenteric and retroperitoneal fat pads were decreased by 20 and 30%, respectively, in the IL-6 treated compared to the saline treated group (FIG. 2B). In line with the decreased weight of dissected fat pads, circulating leptin levels were decreased by 40% in the IL-6 treated group at the end of the study compared to before treatment, as evident from FIG. 2C. The leptin levels of the IL-6 treated group also tended to be lower than in the saline treated group at the end of the study (P=0.054). Leptin levels in the saline treated rats were not significantly decreased during the study.

Serum Chemistry and Behaviour

The levels of insulin-like growth factor-I (IGF-I) in serum did not change from the start to the end of the study and did not differ between the groups (Saline: 975.3±31.1 vs. 923.6±42.9 ng/ml and IL-6: 1012.2±35.5 vs. 949.2±51.1 ng/ml). Insulin levels were not changed during the study in either group and were not different between the groups (Saline: 3.06±0.39 vs. 4.71±1.88 ng/ml and IL-6: 3.76±0.67 vs. 3.43±0.42 ng/ml). Glucose levels were not significantly different between the IL-6 and saline treated groups at the end of the study (Saline vs. IL-6: 274.8±9.6 vs. 248.0±13.4 ng/ml). Corticosterone levels were significantly increased by about 20% after IL-6 treatment (123.3±22.2 ng/ml vs. 204.8±19.4 ng/ml, P<0.01) but not by saline treatment (214.1±38.4 vs. 159.4±33.2 ng/ml). Serum amyloid A (SAA), a sensitive marker for acute-phase reaction was undetectable in both groups at day 0 and day 14 of the study (not shown), indicating that neither treatment induced acute-phase reaction. Neither the saline nor the IL-6 treated rats showed any signs of illness such as staring coat, reduced grooming or discharge from eyes, or reluctance to move. There were no obvious differences in behavior between saline and IL-6 treated rats.

Organ Weights

The relative weights of the heart, liver, kidneys, adrenals and spleen were not affected by IL-6 treatment compared to saline treatment (not shown).

Discussion

This study provides the first demonstration that central treatment with IL-6 reduces adipose tissue mass. The decrease in adipose tissue mass after IL-6 treatment in rats fed a high fat diet was accompanied by a decrease in leptin levels. The average food intake per day measured over the whole two-week study was decreased in the IL-6 treated group. Serum amyloid A, a sensitive marker for acute-phase reaction, was undetectable in all samples and the rats did not show any obvious behavioral changes. Central administration of IL-6 did not affect IGF-I or insulin levels while serum corticosterone levels increased.

IL-6 in high doses over long time may also affect lean body mass and body growth but there is no evidence that IL-6 treatment caused cachexia and illness in the present study. Serum IGF-I levels and the weights of several non-adipose organs were not affected by the ICV IL-6 treatment. Moreover, in this study with daily injections of IL-6, no increase in the acute-phase reactant SAA was observed. Neither were any behavioral alterations associated with illness observed.

The results from the present study show that ICV administration of IL-6 decreases adipose tissue mass in rats fed a high fat diet. Consequently, IL-6 has a physiological and possibly pharmacological role in regulation of body fat at the CNS level.

Experiments Performed on Humans:

Patients and Sampling

Blood samples from obese and lean human subjects were retrieved from another obesity study (in preparation). All the study subjects were recruited in response to advertisements in a local newspaper. The inclusion criteria for the subjects were; male sex, age>18 years and a BMI between 27.5 and 37.0 kg/m² for the obese subjects and below 27.5 kg/m² for the control subjects. The exclusion criteria were: reported weight change of >3 kg in the month prior to examination, diabetes mellitus requiring drug or insulin treatment, cardiovascular disease, unstable smoking, history or presence of eating disorder, or pharmacological treatment with weight-loss agents, antidepressants, steroids, anti-inflammatory drugs or anticonvulsants. There were seven smokers in the obese group and two smokers in the non-obese group. In order to obtain weight stability, all the subjects received individual dietary information and their weight was monitored over a period of three weeks. A total of 34 obese subjects and 10 non-obese subjects were included. All the study subjects were examined after a period of 24 hours at the metabolic ward. Examinations were performed in the morning between 6 and 7.30 am and samples of CSF and serum were taken simultaneously. The sampling of CSF was performed according to standardized procedures with the examined subject in a lateral recumbent position and using a lumbar puncture at the L₃-L₄ or L₄-L₅ interspace with a standard mm needle (Sprotte standard needle with introducer, Ø0.7 mm, 22G, 90/120 mm). After the examinations, the subjects were instructed to rest in a horizontal position for at least 60 minutes. Each sample of CSF was immediately placed on ice and centrifuged at +4° C. Samples were then frozen in separate containers at −80° C. pending analysis. All the subjects gave their written informed consent, and the study protocol was approved by the ethics committee at the University of Goteborg Medical Faculty.

Body Fat

Body composition was measured using a four-scan computed tomography technique (GE high speed advantage) to determine skeletal muscle, subcutaneous and visceral adipose tissue. The following settings were used: 20 kV, 250 mAs, slice thickness 10 mm. Scans were taken at the level of the L₄₋₅ discs. The effective dose equivalent per examination was 0.4-0.8 mSv. The tissue areas and anatomic boundaries were determined as described previously (Chowdhury B, Sjostrom L, Alpsten M, Kostanty J, Kvist H, Lofgren R, 1994, “A multicompartment body composition technique based on computerized tomography” Int J Obes Relat Metab Disord, 18: 219-34). The CV for the determination of subcutaneous adipose tissue was 0.5%, while it was 1.2% for visceral adipose tissue.

ELISA (Enzyme-Linked Immunosorbent Assay)

For measurements of CSF and serum IL-6 levels, the Quantikine High Sensitivity human IL-6 ELISA with a detection limit of 0.156 pg/ml was used (R&D Systems, Minneapolis, Minn.). The assays were used according to the manufacturer's instructions.

Statistical Analysis

Values are given as means and confidence intervals, Comparisons between two groups were made using Student's t-test. Relationships between continuous variables were analyzed using linear regression models. In some cases, a logarithmic transformation was applied to the dependent variable in order to obtain a linear regression relationship. Due to heteroskedasticity, robust standard errors for the regression estimates were calculated according to White's correction (Greene W, 1993, “Econometric Analysis” Second edition vol. New York; MacMillan). The data were analyzed using the Stata statistics package (Stata-Corp. Stata Statistical Software: Release 7.0. College Station, Tex.) and Origin (Microcal Software Inc., Version 6, Northhampton, Mass.).

### Example 3

(Correlation between levels of IL-6 in CSF and Total Body Fat and Subcutaneous Fat)

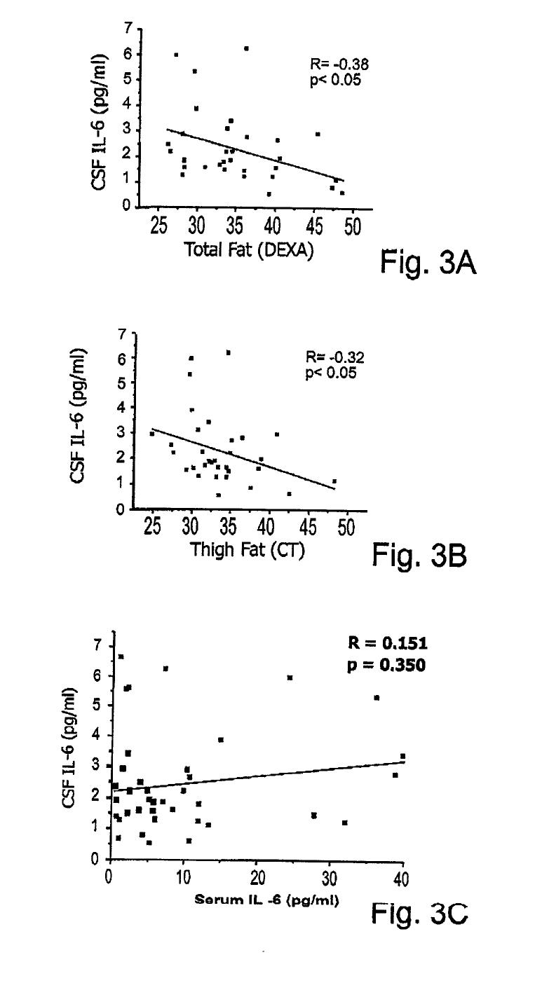

IL-6 levels were measured in CSF samples from a group of obese subjects (BMI 29.1-36.3). There was a clear negative correlation between CSF IL-6 and total body fat measured by DXA as can be seen in FIG. 3A. Furthermore, CSF IL-6 levels were found to correlate negatively with thigh subcutaneous fat measured by CT (FIG. 3B).

These results demonstrate that CSF IL-6 correlated negatively with both total body fat mass and subcutaneous fat, suggesting that body fat regulating neurons in the hypothalamus are exposed to low levels of IL-6 in obese individuals.

### Example 4

(Correlation Between IL-6 in CSF and Serum)

In this example, the correlation between IL-6 levels in the CSF and serum IL-6 levels was studied. IL-6 levels in the CSF showed no correlation with serum IL-6 levels (FIG. 3C). Like leptin, IL-6 is released into the blood circulation from fat and the levels in the circulation correlate positively with BMI (Vgontzas A N, Papanicolaou D A, Bixler E O, Kales A, Tyson K, Chrousos G P, 1997, “Elevation of plasma cytokines in disorders of excessive daytime sleepiness: role of sleep disturbance and obesity”, J Clin Endocrinol Metab 82: 1313-6; Mohamed-Ali V, Goodrick S, Rawesh A, Katz D R, Miles J M, Yudkin J S, Klein S, Coppack S W, 1997, “Subcutaneous adipose tissue releaser interleukin-6, but not tumor necrosis factor-alpha, in vivo”, J Clin Endocrinol Metab 82; 4196-200). However, the present results indicate that CSF IL-6 does not reflect the transport of serum IL-6 across the blood-brain barrier in a similar way as that assumed for leptin (Caro J F, Kolaczynski J W, Nyce M R et al, 1996, “Decreased cerebrospinal-fluid/serum leptin ratio in obesity: a possible mechanism for leptin resistance” Lancet 348: 159-61; Schwartz M W, Peskind E, Raskind M, Boyko E J, Porte D, 1996, “Cerebrospinal fluid leptin levels: relationship to plasma levels and to adiposity in humans” Nat Med 2: 589-93). Instead, there may be an independent regulation of IL-6 in the CNS, by local production in the CNS.

Thus, the present inventors have found that CSP IL-6 correlates negatively with total body fat mass as well as subcutaneous fat mass, the major part of all fat in humans. The data also show that CSF IL-6 is not associated with serum IL-6, suggesting that CSF IL-6 reflects a local regulation, most probably the local production of IL-6 in the CNS, rather than the transport of circulating fat derived IL-6 into the brain. Taken together with the above data that IL-6 suppress fat mass at the CNS level in rodents, these results are in line with the assumption that the target neurons of this anti-obesity effect are exposed to insufficient levels of IL-6 in obese subjects.

1. A method for chronic treatment of obesity wherein a pharmaceutically effective amount of a substance that upon administration to a patient without complete IL-6 deficiency will lead to an increased level of an IL-6 receptor agonist is administered to said patient for reducing adipose tissue mass.

2. A method according to claim 1, wherein said administration will lead to an increased level of an IL-6 receptor agonist in the CSF.

3. A method according to claim 1, wherein said patient has normal levels of IL-6 in serum.

4. A method according to claim 2, wherein said patient has normal levels of IL-6 in serum.

5. A method according to claim 1, wherein said substance is an IL-6 receptor agonist.

6. A method according to claim 2, wherein said substance is an IL-6 receptor agonist.

7. A method according to claim 3, wherein said substance is an IL-6 receptor agonist.

8. A method according to claim 4, wherein said substance is an IL-6 receptor agonist.

9. A method according to claim 1, wherein said substance is IL-6 or a functionally equivalent analogue thereof.

10. A method according to claim 2, wherein said substance is IL-6 or a functionally equivalent analogue thereof.

11. A method according to claim 3, wherein said substance is IL-6 or a functionally equivalent analogue thereof.

12. A method according to claim 4, wherein said substance is IL-6 or a functionally equivalent analogue thereof.

13. A method according to claim 1, wherein said substance is formulated in a form enabling passage of said IL-6 receptor agonist through the blood-brain barrier.

14. A method according to claim 1, wherein said obesity is visceral or general obesity that is due to genetic predisposition.

15. A method according to claim 1, wherein said obesity is caused by lifestyle or environment.

16. A method for chronic treatment of obesity comprising the steps of
administering to a patient without complete IL-6 deficiency a pharmaceutically effective amount of a substance that upon administration to said patient will lead to an increased level of an IL-6 receptor agonist for reducing adipose tissue mass, and
administering to said patient a conventional medicinal product for the treatment of obesity.

17. Use of a substance that upon administration to a patient without complete IL-6 deficiency will lead to an increased level of an IL-6 receptor agonist for the production of a medicinal product for reducing adipose tissue mass for chronic treatment of obesity.

18. Use according to claim 17, wherein said administration will lead to an increased level of an IL-6 receptor agonist in the CSF.

19. Use according to any one of the claims 17-18, wherein said patient has normal levels of IL-6 in serum.

20. Use according to any one of the claims 17-19, wherein said substance is an IL-6 receptor agonist.

21. Use according to any one of the claims 17-20, wherein said substance is IL-6 or a functionally equivalent analogue thereof.

22. Use according to any one of the claims 17-21, wherein said substance is formulated in a form enabling passage of said IL-6 receptor agonist through the blood-brain barrier.

23. Use according to any one of the claims 17-22, wherein said obesity is visceral or general obesity that is due to genetic predisposition.

24. Use according to any one of the claims 17-23, wherein said obesity is caused by lifestyle or environment.
